(19) United States
(12) Patent Application Publication
(10) Pub. No.: US 2007/0049786 A1
(43) Pub. Date: Mar. 1, 2007

(54) Safety-link brachytherapy catheter

(75) Inventor: Gregory K. Edmundson, Rough and Ready, CA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: CYTYC CORPORATION, Marlborough, MA (US)

(21) Appl. No.: 11/215,764

(22) Filed: Aug. 30, 2005

(51) Int. Cl. A61N 5/00; A61M 36/00; A61M 29/00

(52) U.S. Cl. 600/7; 604/104

(57) ABSTRACT

Devices and methods are provided for creating a closed delivery pathway for a radioactive source. In one aspect, the device can be a tube having a connector adapted to mate with an afterloader on its proximal end and a closed distal end portion that can be inserted into a treatment applicator, to effect formation of the closed delivery pathway. The tube can also include an applicator connector disposed between its proximal and distal ends for removably connecting to the treatment applicator.

### FIELD OF THE INVENTION

The present invention relates generally to devices and methods for use in treating proliferative tissue disorders, and more particularly to devices and methods for the treatment of such disorders by the application of radiation.

### BACKGROUND OF THE INVENTION

Malignant tumors are often treated by surgical resection of the tumor to remove as much of the tumor as possible. Infiltration of the tumor cells into normal tissue surrounding the tumor, however, can limit the therapeutic value of surgical resection because the infiltration can be difficult or impossible to treat surgically. Radiation therapy can be used to supplement surgical resection by targeting the residual malignant cells after resection, with the goal of sterilizing them, reducing the rate of recurrence, or delaying the time to recurrence. Radiation therapy can be administered through one of several methods, or a combination of methods, including permanent or temporary brachytherapy implants, and external-beam radiation.

Brachytherapy refers to radiation therapy delivered by a spatially confined source of therapeutic rays inserted into the body at or near a tumor or other proliferative tissue disease site, and can be used for treating malignant brain and breast tumors, among others. For example, brachytherapy is most appropriate where 1) malignant tumor regrowth occurs locally, within 2 or 3 cm of the original boundary of the primary tumor site; 2) radiation therapy is an effective treatment for controlling the growth of the malignant tumor; and 3) there is a radiation dose-response relationship for the malignant tumor, but the dose that can be given safely with conventional external beam radiotherapy is limited by the tolerance of normal tissue. In brachytherapy, radiation doses are highest in close proximity to the radiotherapeutic source, providing a higher dose in the tissue under treatment while sparing surrounding normal tissue.

There are three types of brachytherapy, high dose rate, low dose rate and permanent. Permanent brachytherapy includes the permanent implantation of radioactive “seeds” of palladium or iodine into the tumor or organ tissues. The implanted seeds give off radiation over a period of several months and remain in the organ permanently. Low dose rate (LDR) brachytherapy refers to placement of multiple sources (similar to seeds) in applicators or catheters, which are themselves implanted in a patient's body. These are left in place continuously over a treatment period of several days, after which both the sources and applicators are removed. High dose rate brachytherapy (HDR), uses catheters or applicators similar to those used for LDR. Typically, only a single radiation source is used, but of very high strength. This single source is remotely positioned within the applicators at one or more positions, for treatment times which are measured in seconds to minutes. The treatment is divided into multiple sessions (‘fractions’), which are repeated over a course of a few days. In particular, an applicator or treatment catheter (s) are inserted into the treatment site so that the distal region is located at the treatment site while the proximal end of the applicator protrudes outside the body. The proximal end is connected to a transfer tube, which in turn is connected to an afterloader to create a closed transfer pathway for the radiation source to traverse. Once the closed pathway is complete, the afterloader directs its radioactive source (which is attached to the end of a wire controlled by the afterloader) through the transfer tube into the treatment applicator for a set amount of time. When the treatment is completed, the radiation source is retracted back into the afterloader, and the transfer tube is disconnected from the applicator.

During treatment it is imperative that the pathway between the transfer tube and the applicator remain closed in case the source, or a portion thereof, disengages from the wire (it is also important to completely isolate the source and its wire from fluids in the applicator or body fluids). If the source were to fall off of the wire during treatment, or some other emergency were to occur, the entire transfer pathway, e.g., the transfer tube and the applicator or treatment catheter, must be removed from the patient. Removal of the source and applicator from the patient must be accomplished quickly and may involve invasive procedures to remove the applicator. While this is necessary to prevent harm to the patient, it is burdensome to the treatment staff, as it requires that a physician and physicist always be present during treatment.

Accordingly, there remains a need for improved methods and devices for treating proliferative tissue disorders, and more particularly for the treatment of such disorders by the application of radiation.

### SUMMARY OF THE INVENTION

The present invention provides various devices and methods for use in treating tissue disorders by the application of radiation or other therapeutic rays. In one aspect, the invention includes a transfer catheter that provides a closed delivery pathway for an energy source. The transfer catheter includes a tube having proximal and distal ends where the proximal end of the tube has a connector formed thereon for coupling to an afterloader, and the distal end is closed, thereby creating a closed pathway between the afterloader and the distal end of the tube for receiving the radioactive source. The transfer applicator can also include an applicator connector disposed on the tube between its proximal and distal ends and facing distally for connecting to a treatment applicator.

While the applicator connector can have a variety of configurations, in one embodiment, it can include a male connector that is adapted to couple to a female connector formed on a proximal end of the treatment applicator. The applicator connector can also be adapted to be activated by an instrument, such as locking forceps, such that the portion of the tube distal to the applicator connector can be removed from within the treatment applicator. Alternatively, the applicator connector can be manually activated.

The tube can also include a variety of features to facilitate administration of radiation to a patient. In one embodiment, the tube can include a spring formed within a wall of thereof that is adapted to provide support thereto. Alternatively, the tube can include a plurality of measuring bands formed thereon and placed at intervals relative to one another such that the tube can be used as a radiographic marker. The distal end of the tube can also be configured for placement within the treatment applicator.

In another aspect, a radiation treatment system is provided that includes a treatment applicator and a transfer tube. The treatment applicator can have a distal end adapted to be inserted at a treatment site within a patient, and a proximal end adapted to extend outside the patient's body. The proximal end of the transfer tube can be coupled to an afterloader, and the distal end of the transfer tube can be inserted through the proximal end of the treatment applicator. The distal end of transfer tube can include a closed end portion such that, when the transfer tube is coupled to the treatment applicator, a closed delivery pathway is created between the afterloader and the distal end of the transfer tube. While the transfer tube can be coupled to the treatment applicator in a variety of ways, in one embodiment, the transfer tube can also include an applicator connector between its proximal and distal ends and facing distally for connecting with the treatment applicator. In the preferred embodiment, the distal end of the transfer tube (up to 40-50 cm of it) is insertable into the treatment applicator with the distal tip of the transfer tube thus extending into the body a distance past which the tissue to be treated is located. This transfer tube is able to be disconnected and removed from the applicator either manually, using tools (e.g., forceps or pliers) or via a remotely actuated mechanism. This embodiment can thus allow the safe and painless removal of a disconnected or stuck source, from the patient's body, without manipulations or invasive procedures requiring a physician's direct intervention.

In another aspect, a method for delivering radioactive treatment to a patient is provided. In one embodiment, the method includes placing a treatment applicator into a patient such that a distal end is positioned at or near a treatment site, and a proximal end extends outward from the patient, coupling a proximal end of a source transfer tube to an afterloader, and inserting a closed distal end of the source transfer tube into the proximal end of the treatment applicator. As a result, a closed pathway is created between the afterloader and the closed distal end of the source transfer tube. The method can further include activating an applicator connector formed between the proximal and distal ends of the source transfer tube, and removing the source transfer tube from the treatment applicator while the closed pathway is maintained.

### BRIEF DESCRIPTION OF THE DRAWINGS

The invention will be more fully understood from the following detailed description taken in conjunction with the accompanying drawings, in which:

FIG. 1 is a schematic of an exemplary applicator as disclosed herein;

FIG. 2 is a perspective view of the applicator of FIG. 1; and

FIG. 3 is a perspective view of a treatment system that includes the applicator of FIGS. 1-2 and a treatment applicator.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Disclosed herein are devices and methods for use in treating tissue disorders by the application of radiation, energy, or other therapeutic rays. While the devices and methods disclosed herein are particularly useful in treating various cancers and luminal strictures, a person skilled in the art will appreciate that the methods and devices disclosed herein can have a variety of configurations, and they can be adapted for use in a variety of medical procedures requiring treatment using sources of radioactive or other therapeutic energy. These sources can be radiation sources such as radio-isotopes, or man-made radiation sources such as x-ray generators. The source of therapeutic energy can also include sources or thermal, radio frequency, ultrasonic, electromagnetic, and other types of energy.

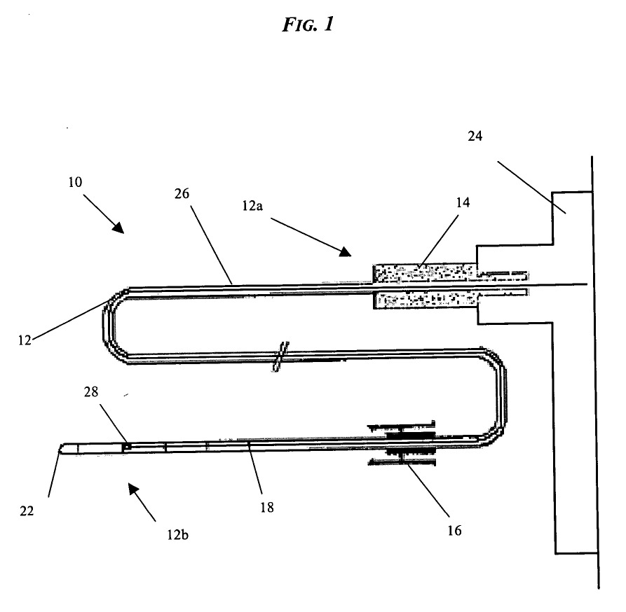

FIGS. 1-3 illustrate one embodiment of a device 10 for providing a closed delivery pathway for a radioactive source. In particular, FIG. 1 illustrates a device 10 for providing a closed delivery pathway for a radioactive source attached to an afterloader device 24 which is configured to load a wire 26 having at least one radioactive source 28 disposed thereon inside the device 10. As shown, the device 10 can include a tube 12 (or applicator or catheter) having a connector adapted to mate with an afterloader on its proximal end 12a and a closed distal end portion 22 that can be inserted into a treatment applicator to effect formation of a closed delivery pathway. The tube 12 can also include an applicator connector 16 disposed thereon between its proximal and distal ends 12a, 12b for connecting to a treatment applicator, as will be discussed in more detail below.

Illustrated tube 12 has a substantially cylindrical shape with proximal and distal ends 12a, 12b, however, a person of ordinary skill will recognize that tube 12 can have a variety of configurations, such as cross-sectional shapes that are circular, oval, ellipsoid, polygon, kidney-bean shaped, a composite of substantially circular, oval, or ellipsoid forms, or irregularly shaped. At least one lumen can extend from the proximal end 12a to the distal end 12b of the tube 12, and can be adapted to receive a wire 26 that can hold a radioactive source 28 thereon.

The proximal end 12a of the tube 12 can include a connector 14 that is adapted to be inserted into a connection port on an afterloader 24 or that can attach to the exterior thereof. While the connector 14 can have a variety of configurations, exemplary connectors can be snap-fit, quick-connect, Quick-fit™ (Varian Medical Systems) or other types of locking connectors. Alternatively, a variety of male or female sockets or connection hubs can be used, and can further have features such as cut-outs, biasing means, and locks to facilitate mating between the proximal end 12a of the tube 12 and the afterloader 24. One skilled in the art will appreciate the variety of ways that the proximal end 12a of the tube 12 can couple to the afterloader 24. The connector 14 can also include features to facilitate the entry of the radioactive source into the proximal end 12a of the tube 12, such as, for example, a tapered or funnel opening formed thereon.

The distal end 12b of the tube 12 can be adapted to form a closed pathway between the afterloader and the distal end 12b of the tube 12 when it is coupled to a treatment applicator 41. In one embodiment, the distal end 12b of the tube 12 can include a closed end portion 22. While the closed end portion 22 can be effected in any way known in the art, in one exemplary embodiment, the distal end 12b of the tube 12 can include a reinforced plug or a variety of other sealing agents. The distal end 12b of the tube 12 can also be adapted to facilitate placement into a treatment applicator, and can be rounded or include a slight distal taper.

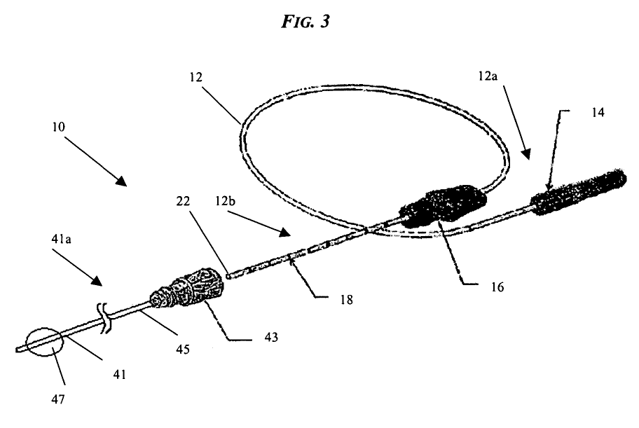

An applicator connector 16 that is adapted to removably connect to a treatment applicator (such as treatment applicator 41 shown in FIG. 3) can be located between the proximal and the distal ends 12a, 12b of the tube 12. The applicator connector 16 effectively divides the tube 12 into two portions. The proximal portion of tube 12 between the proximal end 12a and the applicator connector 16 can act as a transfer tube between the after loader and the applicator for loading the radioactive source 28 from the afterloader 24 into the applicator. The distal portion of tube 12 between the applicator connector 16 and the distal end 12b is adapted for insertion into an applicator and provides a closed path within the applicator in which a radioactive source 28 may reside.

In certain embodiments, tube 12 may be a continuous length of tubing from the proximal 12a to the distal end 12b. In other embodiments, tube 12 may be divided into separate sections of tubing as long as tube 12 provides a closed pathway with at least one lumen extending from the proximal 12a to the distal end 12b. For example, tube 12 could be formed as two separate sections of tubing corresponding to the proximal and distal portions described above, with each section of tubing connected to the applicator connector 16, with the applicator connector 16 providing an inner lumen that corresponds with an inner lumen in each portion of the tubing so that a closed pathway and a continuous lumen are provided between the proximal and distal ends 12a, 12b. In certain embodiments, the applicator connector 16 can also be fixedly or movably positioned on the tube 12, depending upon the needs of the user.

One skilled in the art will appreciate that the tube can be formed from a variety of materials, with a preference for flexible materials (e.g., silicone, nylon, etc.). In other examples, all, or a portion, of the transfer tube may be rigid. The chosen material can include both radiation lucent and radiation opaque materials. For example, radio-opaque materials such as barium, tungsten, bismuth, tantalum, and tin can be coated on a surface of the tube 12 to shield sensitive tissue, or to aid in radiographic localization of the transfer tube within the body. As an alternative to coating, a radiation-blocking or absorbing shield (not shown) can be positioned between the radiation source and adjacent tissue. The chosen materials can also have a variety of properties or features that aid in the procedure such as MRI compatibility, and MRI, radiographic or ultrasound contrast properties. The shielding can be in a variety of configurations, including circumferential, partially circumferential, longitudinal and of varying shapes and sizes along the length of the transfer tube.

The tube 12 can also include features to provide support thereto. For example, in one embodiment, the tube can include a spring placed within at least a portion of the walls thereof. Alternatively, a splint or any other piece of metal or plastic configured to provide support to the tube can be placed within or outside of the walls thereof. Such a support might be particularly useful in a region near applicator connector 16, near proximal connector 14, or in the proximal portion of tube 12 generally so as to provide a pathway for the radiation source between the afterloader and the applicator that is generally smooth and kink free.

Tube 12 can include a plurality of measuring bands or markers 18 formed thereon at or near its distal end 12b to assist with locating and/or positioning the applicator within a patient, or measuring the location of the radiation source 28 therein. In an exemplary embodiment, the measuring bands 18 can be placed at intervals relative to one another such that the tube 12 can be used as a visual or radiographic marker. While the measuring bands 18 can be formed at a variety of locations along the tube 12, such as just distal to the applicator connector 16 and extending along the length of the tube 12 to the distal most portion of the distal end 12b, as shown the measuring bands 18 are formed along the length of the distal end 12b of the tube 12.

One skilled in the art will appreciate that a variety of measuring bands can be used with the tube 12. For example, fiducial markers, such as those disclosed in patent application Ser. No. 10/704,161, entitled “Tissue Positioning Systems and Methods for Use with Radiation Therapy,” and incorporated herein by reference in its entirety, can be used. In addition, radiation sensors can be used with or disposed on tube to assist with delivery of therapeutic radiation. Utility application Ser. No. 10/704,340, entitled, “Implantable Radiotherapy/Brachytherapy Radiation Detecting Apparatus and Method,” and incorporated herein by reference in its entirety, discloses exemplary sensors.

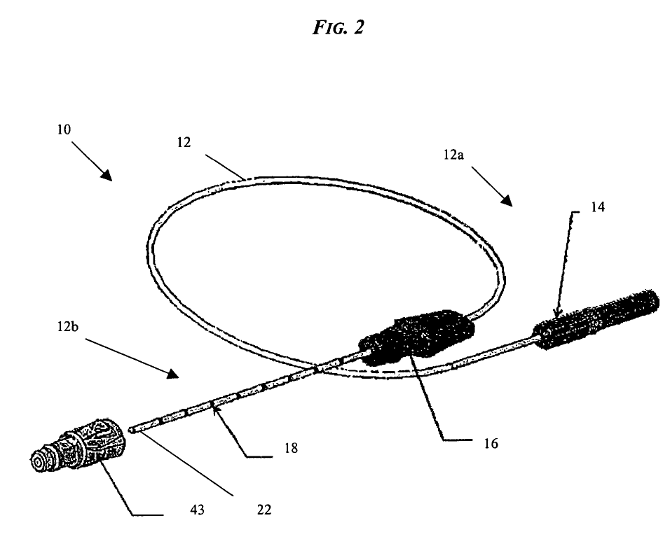

FIG. 2 illustrates tube 10 having an applicator connector 16 oriented for attachment to a proximal connector 43 such as might be found on a treatment applicator such as treatment applicator 41 of FIG. 3. In particular, the applicator connector 16 is oriented so that it faces the distal direction in order to removably attach to the treatment applicator proximal connector 43. This connection can be releasable and can be effected in a variety of ways. For example, the applicator connector 16 can include a male connector formed thereon that is adapted to mate with a corresponding female connector 43 formed on or attached to a proximal end of the treatment applicator. The point of attachment between the treatment applicator and the transfer tube can further be anywhere along the length of the treatment applicator, including a location such as its distal (internal) tip. Any method of positively securing the transfer tube to the applicator (e.g., a force used to insert and retain for the desired time, then the force removed for removal of the transfer tube from the applicator) can be used.

By way of non-limiting example, the connector formed on the applicator connector 16 can include a latch, or a biased or spring loaded latch mechanism. In this way, the distal portion of tube 12 that is adapted for insertion into the applicator can be inserted through an inner lumen of the treatment applicator proximal connector 43, and tube 12 slid distally with respect to the connector 43 until the connectors 16, 43 engage. Where a biased latch mechanism is deployed, connectors 43, 16 may lock together simply by sliding one into the other. For releasing the locked connection, the latch can be adapted to be operated manually by a user, or alternatively, using an instrument, such as clamps, hemostats, pliers, or forceps such that when activated, connectors 43, 16 become unlocked and the tube 12 can be removed from the treatment applicator. While the exemplary embodiment illustrates a latch mechanism, one skilled in the art will appreciate that a variety of other ways can be used to removably connect the device 10 to the treatment applicator.

FIG. 3 illustrates device 10 with a treatment applicator 41. While a variety of treatment applicators can be used, in one exemplary embodiment treatment applicators as disclosed in U.S. Pat. No. 5,429,582 to Williams, issued on Jul. 4, 1995 and entitled “Tumor Treatment;” U.S. Pat. No. 6,413,204 to Winkler et al., issued on Jul. 2, 2002 and entitled “Interstitial Brachytherapy Apparatus and Method for Treatment of Proliferative Tissue Diseases;” and U.S. Pat. No. 6,607,477 to Longton et al., issued on Aug. 19, 2003 and entitled “Graduated Intraluminal Catheter and Methods of Use Thereof;” each of which is hereby incorporated by reference herein, can be used. In particular, the treatment applicator 41 can be positioned within the patient such that the distal end is positioned within a patient at or near a treatment site and anchored there by an anchoring element such as balloon 47, and a catheter 45 providing a lumen extends proximally out of the patient's body so that connector 43 is accessible to a surgeon outside of the patient.

Methods for delivering radioactive treatment to a patient are also provided according to embodiments of the invention. Such a method generally begins with the step of placing the treatment applicator 41 within the patient. In certain embodiments, this step includes surgically resecting, incising or otherwise altering a patient's tissue followed by implanting a treatment applicator into the site of the resection cavity. Examples of this type of applicator placement can be found in the previously incorporated U.S. Pat. No. 5,429,582 for the treatment of brain cancers and U.S. Pat. No. 6,413,204 for the treatment of breast cancer. Still further, the placement of a treatment applicator could involve anchoring the treatment catheter in a body lumen, such as disclosed in the previously incorporated U.S. Pat. No. 6,607,477 which uses such applicators to apply radiation to urethral tissue.

Following placement of the treatment applicator 41 within the patient, the proximal end 12a of the tube 12 can be coupled to an afterloader (or other means by which a radioactive or other source of therapeutic rays by be provided) using the connectors discussed above, and the distal end 12b of tube 12 can be inserted into the proximal end 41a of the treatment applicator 41 to a desired location, as shown in FIG. 3. In particular, the distal end 12b can be inserted into the catheter 45 of applicator 41 until the proximal connector 43 of the applicator engages the applicator connector 16 on tube 12.

With the applicator implanted and the device 10 connected to the applicator 41 and the afterloader, a radioactive source can be delivered by the afterloader through tube 12 so as to dwell within the applicator for a desired or prescribed period of time.

If the need arises to remove the remove the radioactive source from the patient while maintaining a closed pathway for the source, device 10 allows for the rapid removal of the source without the need to remove the implanted applicator. In particular, the applicator connector 16 can removed from the treatment applicator 41 while the closed radioactive source pathway is maintained by tube 12 having a closed distal end 22. In order to effect removal, the applicator connector 16 on tube 12 can be decoupled from the connector 43 located on the treatment applicator 41. For example, in one exemplary embodiment where the applicator connector 16 has a latch mechanism, the latch can be activated using an instrument or manually. As a result, the device 10 can be separated from and withdrawn from the treatment applicator 41 while leaving the treatment applicator 41 positioned within the patient. This approach advantageously allows for the removal of the radiation source from the patient while maintaining a closed pathway for the radiation source without the need to remove treatment applicator 41.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A device for providing a closed delivery pathway for an energy source, comprising:
a tube having proximal and distal ends;
a connector formed on the proximal end of the tube adapted to couple to an afterloader;
a closed end formed on the distal end of the tube such that a closed pathway for receiving a radioactive source is created between the afterloader and the distal end of the tube; and
an applicator connector disposed on the tube between its proximal and distal ends and facing distally for connecting to a treatment applicator, wherein the portion of the tube distal to the applicator connector is configured for placement within the treatment applicator.

2. The device of claim 1, wherein the tube includes a plurality of measuring bands formed therein.

3. The device of claim 2, wherein the measuring bands are placed at intervals relative to one another such that the tube can be used as a radiographic marker.

4. The device of claim 1, wherein the applicator connector includes a male connector that is adapted to couple to a female connector formed on a proximal end of the treatment applicator.

5. The device of claim 1, wherein the applicator connector can be activated by an instrument such that the portion of the tube distal to the applicator connector can be removed from within the treatment applicator.

6. The device of claim 5, wherein the instrument is a locking forceps.

7. The device of claim 1, wherein the applicator connector can be manually activated such that the portion of the tube distal to the applicator connector can be removed from within the treatment applicator.

8. The device of claim 1, wherein the tube includes a spring formed within a wall of thereof that is adapted to provide support thereto.

9. The device of claim 1, further comprising a source of therapeutic energy disposed within the tube.

10. The device of claim 9, wherein the source of therapeutic energy includes a radioactive source.

11. The device of claim 9, wherein the source of therapeutic energy includes a man-made radiation source.

12. A radiation treatment system, comprising:
a treatment applicator having a distal end adapted to be inserted at a treatment site within a patient, and a proximal end adapted to extend outside the patient's body; and
a transfer tube having a proximal end adapted to couple to an afterloader, and a portion of its closed distal end placed within the proximal end of the treatment applicator, wherein a closed pathway is formed between the transfer tube and the afterloader when the transfer tube is coupled to the treatment applicator.

13. The system of claim 12, wherein the transfer tube further includes an applicator connector disposed between its proximal and distal ends and facing distally for connecting with the treatment applicator.

14. The system of claim 13, wherein the applicator connector is adapted to be activated by an instrument such that the portion of the tube distal to the applicator connector can be removed from within the treatment applicator.

15. The system of claim 14, wherein the instrument is a forceps.

16. The system of claim 13, wherein the applicator connector is adapted to be activated manually such that the portion of the tube distal to the applicator connector can be removed from within the treatment applicator.

17. The system of claim 12, wherein the transfer tube includes a spring formed within a wall thereof that is adapted to provide support thereto.

18. The system of claim 12, wherein the transfer tube includes a plurality of measuring bands formed thereon.

19. The system of claim 18, wherein the measuring bands are placed at intervals relative to one another such that the transfer tube can be used as a radiographic marker.

20. The system of claim 12, further comprising a radiation source disposed within the portion of the transfer tube that is disposed within the treatment application.

21. The system of claim 12, further comprising an afterloader having a radiation source connected to a proximal portion of the transfer tube.

22. A method for delivering radiation treatment to a patient, comprising:
placing a treatment applicator into a patient such that a distal end is positioned at or near a treatment site, and a proximal end extends outward from the patient;
coupling a proximal end of a source transfer tube to an afterloader; and
inserting a closed distal end of the source transfer tube into the proximal end of the treatment applicator, such that a closed pathway is created between the afterloader and the closed distal end of the source transfer tube.

23. The method of claim 22, further comprising activating an applicator connector formed between the proximal and distal ends of the source transfer tube, and removing the source transfer tube from the treatment applicator while the closed pathway is maintained.

24. The method of claim 23, wherein the applicator connector is activated by manipulation thereof with an instrument.

25. The method of claim 23, wherein the applicator connector is activated by a user.

26. The method of claim 22, further comprising operating the afterloader to dispose a radiation source within the closed distal end of the transfer tube.
